(19) United States
(12) Patent Application Publication
(10) Pub. No.: US 2004/0102732 A1
(43) Pub. Date: May 27, 2004

(54) Dialysis system for treatment of vulnerable patients and methods of use

(76) Inventors: Morteza Naghavi, Houston, TX (US); Hossein Eftekhari, Tehran (IR); S. Ward Casscells, Houston, TX (US)

Correspondence Address: Gary R. Maze, Duane Morris LLP, Suite 500, One Greenway Plaza, Houston, TX, 77046, US

(21) Appl. No.: 10/465,200

(22) Filed: Jun. 19, 2003

Related U.S. Application Data
(60) Provisional application No. 60/389,834, filed on Jun. 19, 2002.

(51) Int. Cl. A61M 1/00

(52) U.S. Cl. 604/29

(57) ABSTRACT

The present invention relates generally to a system and methods for local delivery of drugs directly to the coronary circulation which may be isolated from systemic circulation. More especially it relates to a dialysis system and methods of infusing beneficial drugs, therapeutic agents, and/or other beneficial substances, including high doses of these, such as HDL, therapeutic genes, and/or chelating agents to the coronary system. The multi-chambered dialysis machine in the present system is capable of removing unwanted/harmful substances from the blood, enriching and/or otherwise processing the blood, and re-circulating the processed blood back to the coronary circulation of patient. It is emphasized that this abstract is provided to comply with the rules requiring an abstract which will allow a searcher or other reader to quickly ascertain the subject matter of the technical disclosure. It is submitted with the understanding that it will not be used to interpret or limit the scope or meaning of the claims.

### RELATION TO PRIOR APPLICATIONS

The present invention claims priority through U.S. Provisional Application No. 60/389,834, filed Jun. 19, 2002.

### FIELD OF INVENTION

The present invention relates generally to the field of coronary medical devices. More specifically, the present invention discloses a catheter-based coronary dialysis system that is useful for providing therapies to treat atherosclerosis and related diseases.

### BACKGROUND OF THE INVENTION

Every year about 1.5 million people in the United States have heart attacks, and more than half of them die. Vulnerable plaque as the major underlying cause of acute coronary syndromes and sudden cardiac death is now the focus of interest in cardiovascular medicine. The fast growing body of knowledge about atherosclerosis and the pressing need for early detection and treatment of patients at risk of fatal heart attacks has led to the emergence of the new field of “vulnerable plaque.” In the past several years, the field of vulnerable plaque research has evolved in a rapid and progressive way. As the understating of the culprit lesions is changing, terminologies and criteria for definitions and classifications need to be revised and updated.

Several kinds of clinical observations suggested that instead of progressive growth of the intimal lesion to a critical stenosis, complication of a not necessarily occlusive plaque by thrombosis most often causes episodes of acute coronary syndrome. It is now appreciated that physical disruption of the atherosclerotic plaque commonly causes acute thrombosis.

The two major modes of plaque disruption provoke most coronary thrombi. The first mechanism, accounting for some two thirds of acute coronary syndrome (“ACS”), involves the fracture of the plaque's fibrous cap. The second mode involves a superficial erosion of the intima.

Atherosclerosis, formerly considered a lipid storage disease, actually involves an ongoing inflammatory response. Substantial advances in basic and experimental science have illuminated the role of inflammation and the underlying cellular and molecular mechanisms that contribute to atherogenesis. Recent advances in basic science have established a fundamental role for inflammation in mediating all stages of this disease from initiation through progression and, ultimately, the thrombotic complications of atherosclerosis. These new findings provide important links between risk factors and the mechanisms of atherogenesis. Clinical studies have shown that this emerging biology of inflammation in atherosclerosis applies directly to human patients.

Current therapies include patient counseling, dietary counseling, pharmacotherapy, life style modification, and surgery. Even with aggressive thrombolytic, anticoagulant, and/or antiplatelet agents or interventional therapy, patients with ACS still have a 12% to 16% incidence of major cardiac events at 4 to 6 months after hospital discharge. Novel treatments based on increased understanding of the underlying mechanisms of plaque instability should yield further improvements in outcomes. Growing evidence indicates that in ACS, elevated circulating inflammatory markers, such as CRP, serum amyloid A, IL-6, and IL-1 receptor antagonist commonly accompany ACS. Such elevations correlate with in-hospital and short-term adverse prognosis and may reflect not only a high prevalence of myocardial necrosis, ischemia-reperfusion damage, or severe coronary atherosclerosis but also a primary inflammatory instigator of coronary instability in particular C-reactive protein (CRP) which predicts an unfavorable course, independent of the severity of the atherosclerotic or ischemic burden. Thus, inflammation represents one potential novel pathophysiological mechanism of the ACS that may furnish such a new target for therapy.

A large number of experimental studies have shown that augmentation of HDL and its apoprotein may have vascular protective, preventive, and therapeutic effects. Conventional treatment of patients suffering from acute coronary syndrome (ACS) consists typically of antiplatelet, anticoagulant, thrombolytic therapy, including percutaneous coronary intervention. Taking in to account the fact that there are at least 2-3 vulnerable plaques in each patient as well as inaccessibility of about 50% of lesions by stents, other strategies are important in stabilizing vulnerable plaques within the coronary circulation. One way a effect may be achieved is by delivering high dose of drugs that are able to stabilize the plaques. Additionally, harmful and other unwanted toxic substances that are involved in atherogenesis, plaque vulnerability, and acute coronary syndrome may be removed from the blood to decrease complications and recurrence rate of acute coronary syndromes as well as increasing the survival of the patients.

Typically, several dozen plaques are found in arteries afflicted by ACS disease. It is the rupture of these plaques that brings about the terminal stage of the disease. The rupture causes a large thrombus to form which may or may not completely occlude the vascular lumen. The importance of diffuse therapy would be clearer by understanding the inaccessibility of nearly 50% of coronary artery plaques by stent.

Cholesterol removal and excretion is at least as important as cholesterol mobilization from peripheral tissues. For example, implementation of any therapeutic strategy that could deliver high dose of HDL in to the target organ (coronary circulation) through systemic administration of drug could facilitate the process of plaque stabilization. On the other hand because HDL therapy may only mobilize the peripheral cholesterol through reverse cholesterol transport mechanism but may not be able to remove or excrete the cholesterol from the body. Current LDL aphaeresis machine using plasmaphaeresis and selective LDL aphaeresis is able to precipitate LDL and a limited number of plasma harmful factors but adding the other technique such as immunprecipitation, selective absorption, and filtration to the current LDL aphaeresis technology will increase the capabilities to decrease more harmful factor as possible. By combining the LDL aphaeresis machine together with a system that is able to deliver high dose of HDL to the circulation as well as coronary circulation, we would be able not only mobilizing the cholesterol from the peripheral tissues but also we are able to remove and facilitate the excretion of the effluxed cholesterol out of the body.

On the other hand by addition of other technique to the established LDL aphaeresis machine such as immunoprecipitation (immunoabsorption) and other precipitating mechanisms would be able to precipitate and filterate more harmful substances for patients with chronic coronary artery disease.

C-reactive protein (CRP) is a trace serum protein which elevates up to 1000-fold in concentration in association with inflammation and tissue necrosis. CRP binds with phosphocholine and phosphate esters; initiates reactions of agglutination, opsonization and complement consumption; and precipitates with protamine and synthetic polymers of lysine and arginine, and these reactivities are modulated by calcium and phosphocholine. There is report on the interactions of heparin with these polycations in the absence and presence of CRP, which show marked similarities to reactions between antigen and antibody. Heparin optimally precipitated with the polycations over a narrow range of reactant ratios, peaking at slight anion charge excess.

Clinical studies affirm correlation of circulating markers of inflammation such as CRP with propensity to develop ischemic events and with prognosis after ACS. Intralesional or extralesional inflammation may hasten atheroma evolution and precipitate acute events. Circulating acute-phase reactants elicited by inflammation may not only mark increased risk for vascular events, but in some cases- may contribute to their pathogenesis. So it may be logical that decreasing the level of inflammatory markers such as CRP may have significant effect on the pathogenesis of atherosclerosis and its complication (ACS). This may be achieved by adding and combining specific chamber to the dialysis machine to precipitate CRP.

Human C-reactive protein is associated with lipids. Isolation of pure lipid-free C-reactive protein was obtained by a three step procedure. First, partially lipid-free C-reactive protein was obtained by affinity chromatography; second, lipid-bound proteins were eliminated by calcium-dependent precipitation; and third, lipid-free pure C-reactive protein was obtained by affinity re-chromatography of the supernatant-4 46-50% yield of lipid-free C-reactive protein was obtained compared with the 14.7% obtained by the old method of extraction with lipid solvents.

Febrile-range temperature induction in this invention relates to the treatment of the harmful inflammation and inflammatory mediators such as cytokines in the body tissue by exposing the inflammatory cells and blood to heat. A plaque is an accumulation of cholesterol, proliferating smooth muscle cells, and inflammatory cells covered by cellular secretion of collagen that formed the cap over the plaque in the vessel wall. Macrophages migrate in to and accumulate in the plaque causing inflammation which causes the plaque prone to rupture and formation of blood thrombus. Rupture typically is caused by inflammatory cells, primarily macrophages. These cells release enzymes that tend to degrade the cap. A number of studies have shown that heat may induce programmed cell death. Heating also causes the melting or de-crystallization of the cholesterol crystal within the plaque. So heating the blood (41-42° C.) within the machine during its passage is another plaque stabilizing method that implemented in this patent in order to decrease the inflammatory process within vulnerable atherosclerotic plaques. On the other side it has been shown that febrile range temperature by itself may modify the profile of plasma level of some cytokine such as TNF-alpha, IL-1, and IL-6 making it logical to speculate that heating the blood to the febrile range level may have some useful effect in reducing the level of some cytokine especially in acute coronary syndrome as well as stabilizing the vulnerable plaques.

In general, coronary dialysis system 1 may be used for separating harmful substances such as LDL, fibrinogen, C reactive protein, and cytokines from circulation. Coronary dialysis system 1 may deliver high level of HDL locally and maintain appropriate plasma level of HDL to reduce its unwanted side effects. This system is also capable of maintaining blood oxygenation, (even delivering hyperbaric oxygen to coronary artery system), sustaining appropriate coronary perfusion pressure and distributing blood with increased temperature.

Systemic coronary dialysis system is provided for performing dialysis of the blood of patients suffering from atherosclerosis in order to reduce the rate of the progression of atherosclerotic plaques, reducing the vulnerability of plaques by compositional changes in the plaque, and even inducing plaque regression. This system designed mainly on the basis of reducing coronary artery disease risk factors especially those which are resistant to the conventional therapeutic modalities or for those there has not been any proven and effective treatment, such LDL aphaeresis and adding the useful factors to the circulation which may have beneficial effect in plaque stabilization.

Systemic drug therapy has a problem of drug dilution that may decrease the effectiveness of the treatment and raise the issue of hazard systemic side effects. In contrast, local therapeutic modality and local drug and/or gene delivery would provide more effective treatment to affect the process of atherogenesis and stabilize atherosclerotic plaques.

### BRIEF DESCRIPTION OF THE DRAWINGS

FIG. 1 is a schematic overview of an exemplary system;

FIG. 1A is a schematic overview of a multi-chambered dialysis machine in situations where the cardiac circulatory system is completely isolated from systemic circulation, e.g. by an inflatable balloon at the tip of the perfusion catheter so that oxygenation of the processed blood is required;

FIG. 1B is a schematic overview of a multi-chambered dialysis machine 100 in cases where the step of oxygenation is not required;

FIG. 2 is a plan view in partial cutaway of two perfusion or dialysis catheters within an arterial shield;

FIG. 3 is a plan view of two perfusion or dialysis catheters within a vehicle catheter;

FIG. 3a is a cross section of a vehicle catheter exemplar showing two perfusion catheters disposed within as well as a pressure or vacuum channel;

FIG. 4a is a cross section of a perfusion catheter showing a temperature channel, pressure channel, and inflation channel;

FIG. 4b is a cross section of a perfusion catheter, collection catheter, or vehicle catheter with a balloon configuration to substantially occlude the vessel into which the catheter is placed;

FIG. 4c is a cross section of a perfusion catheter, collection catheter, or vehicle catheter with a ring balloon configuration which will not completely occlude the vessel into which the catheter is placed;

FIG. 4d is a cross section of a perfusion catheter, collection catheter, or vehicle catheter with a butterfly balloon configuration which will not completely occlude the vessel into which the catheter is placed;

FIG. 5a is a schematic of a coronary system using femoral entry;

FIG. 5b is a plan view in partial cutaway showing two perfusion catheters disposed through the aorta into two separate vessels;

FIG. 5c is a partial perspective view of a collection catheter and arterial shield where the collection catheter is placed into a coronary sinus;

FIG. 6a is similar to FIG. 5a but illustrates use of an aortic ring balloon;

FIG. 6b is a partial cutaway showing a vehicle catheter with an aortic root ring;

FIG. 6B illustrates cross sections from proximal toward distal end of a vehicle catheter with an aortic root ring;

FIG. 7 illustrates the pathways of perfusion catheters and collecting catheter inserted through the femoral artery and the left subelavian vein respectively;

FIG. 8a illustrates the pathways of perfusion catheters and collecting catheter inserted through the femoral artery and femoral vein respectively;

FIG. 8b illustrates the collection of blood in the right atrium by a collecting catheter introduced through the femoral vein;

FIG. 9 illustrates a non-coronary, systemic configuration; and

FIG. 10 illustrates a pericardial configuration.

### DETAILED DESCRIPTION OF THE INVENTION

As used herein, “catheter” is either a general term as will be understood by those of ordinary skill in the medical arts or a specific type of catheter, as understood from the context.

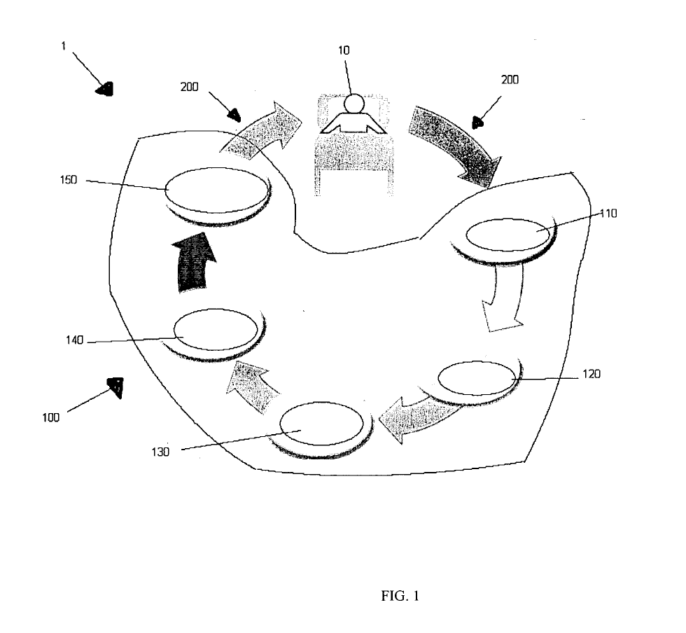

Referring now to FIG. 1, coronary dialysis system 1 comprises multi-chambered dialysis machine 100 and catheter system 200. Patient 10 is connected to coronary dialysis system 1 using catheter system 200 and blood routed through multi-chambered dialysis machine 100 to ameliorate blood components. The apparatus and methods disclosed and claimed herein may be used to deliver drugs, including continuous delivery of a high dose of drugs such as cholesterol removing drugs, locally into the coronary system of patient 1. These may be used to stabilize vulnerable plaques, decrease the lipid content of the plaques, reduce inflammatory activity throughout the coronary system, change the cellular composition of the plaques by decreasing the macrophages and increasing the smooth muscle cells, and the like, or combinations thereof.

In addition to perfusion of doses, including high doses, of substances such as high density lipoprotein (HDL), gene therapy may be introduced directly to the coronary circulation using coronary dialysis system 1. Harmful and/or unwanted plasma substances may be withdrawn from the circulation, e.g. by plasmaphaeresis and aphaeresis in which various separation methods such as precipitation, filtration, adsorption, and immunprecipitation (immunoabsorption). These harmful substances may include LDL, CRP, fibrinogen, LP(a), tissue factor, CD14, interleukin-1, interleukin-6, TG, plasminogen, complement components C3, C4, C 1 inhibitor, and the like, or combinations thereof. Warming the blood during its passage through multi-chambered dialysis machine 100, e.g. using oxygenator 120 or heater 140, by itself may reduce inflammatory process within the plaques, decrease the vulnerability of the plaques and decrease the level of some plasma cytokines such as TNF-alpha, IL-1, and IL-6. During processing, the homodynamic status of patient 10 may be under intensive control.

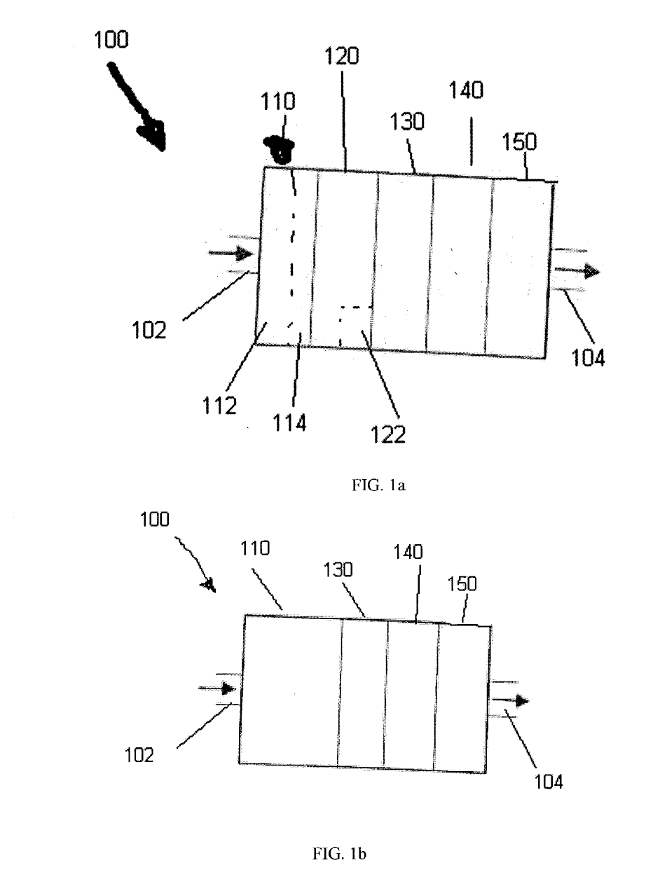

FIG. 1a illustrates an exemplary configuration of multi-chambered dialysis machine 100 in situations where the cardiac circulatory system is to be completely isolated from systemic circulation, e.g. by use of inflatable balloon 222 (FIG. 2) at tip 221 (FIG. 2) of perfusion catheter 220 (FIG. 2). Such isolation requires oxygenation of the processed blood, e.g. via oxygenator 120.

FIG. 1b illustrates an exemplary configuration of multi-chambered dialysis machine 100 in cases where the step of oxygenation is not required.

Referring back to FIG. 1a, multi-chambered coronary dialysis machine 100 comprises fluid inlet 102, fluid outlet 104, and a plurality of chambers 110-150. Each chamber 110-150 may be designed for a specific purpose. Further, as used herein, each chamber 110-150 may be physically separate from at least one other chamber 110-150, all other chambers 110-150, or combined with one or more other chambers 110-150. For example, heater 140 may be a separate chamber, included as part of oxygenator 120, or both.

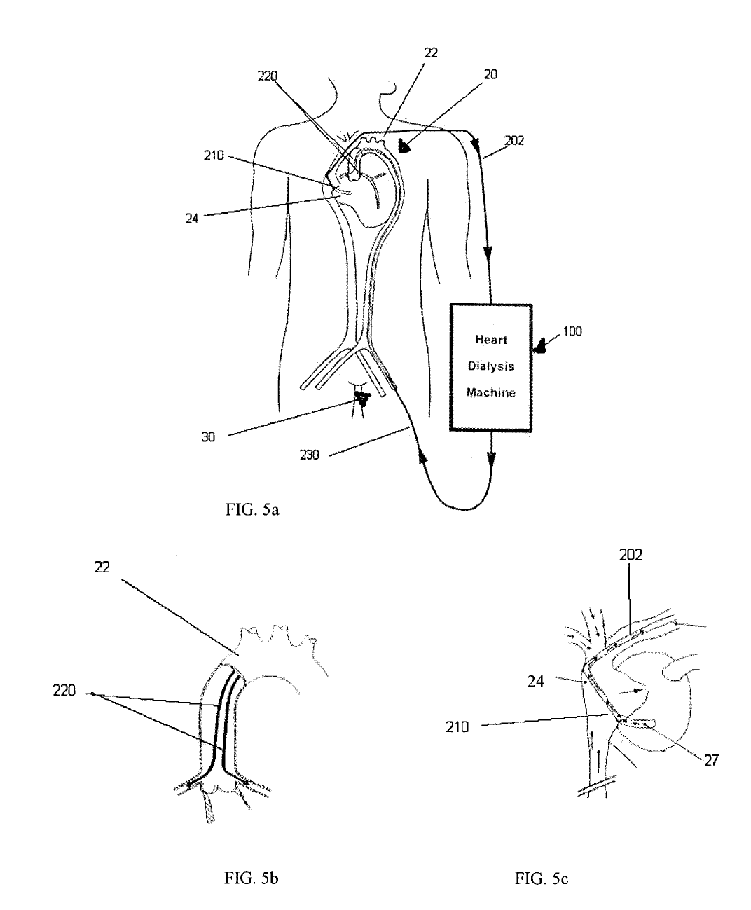

Referring back to FIG. 1, coronary dialysis system 1 may be used to provide stabilization, including rapid stabilization, of vulnerable plaque. It may be appreciated that catheter system 200 (comprising perfusion catheter 220 (FIG. 2) and collecting catheter 210 (FIG. 5a)) and multi-chambered dialysis machine 100 may be used to form a substantially closed circulatory pathway for a fluid such as a patient's blood. In an embodiment, blood is collected such as through collecting catheter 210, drained through fluid inlet 102 (FIG. 1a) of multi-chambered dialysis machine 100, and passed through several chambers 110-150 in which the blood may be filtered, precipitated, enriched, and pumped back through fluid outlet 104 (FIG. 1b) of multi-chambered dialysis machine 100 to the coronary circulation of patient 10 via perfusion catheter 220.

Blood separation chamber 110 may be used to separate blood plasma from blood cells. Blood separation chamber 110 is in fluid communication with fluid inlet 102 (FIG. 1a) of multi-chambered dialysis machine 100. It is understood that blood separation chamber 110 may comprise one or more chambers, e.g. plasmaphaerersis chamber 112 (FIG. 1a) and aphaeresis chamber 114 (FIG. 1a). Further, in an embodiment, blood separation chamber 110 comprises either a plasmaphaerersis (primary separation) chamber, an aphaeresis (plasma differential separation), or both, either as separation sub-chambers or as a single chamber.

Plasma may then be further processed by secondary and/or selective precipitation and filtration to remove undesired substances from the plasma, e.g. harmful and unwanted substances. These harmful and unwanted substances may include intrinsic particles like LDL, CRP, fibrinogen, or any added materials to the perfused blood such as high level of genes, drugs, and/or chelating agent. Blood separation chamber 110 may further comprise immune precipitation functionality to specifically precipitate and separate any plasma harmful factors to further decrease their plasma level.

In the blood separation chamber 110, blood cells are temporarily separated from plasma. During aphaeresis of plasma components, LDL, CRP, fibrinogen, some plasma cytokines, chelating agents, and transgenic material may be separated such as through precipitation, filtration, and/or adsorption so that at the end of this stage of plasma processing, much toxic or harmful substances are removed from the plasma before it is admixed with its blood cells.

In an exemplary embodiment, oxygenator 120 comprises a gas exchanger in which the withdrawn deoxygenated venous blood is oxygenated to an appropriate level of oxygenation for use as natural arterial blood appropriate to be perfused directly into the coronary arterial system of patient 10. During the oxygenation process within oxygenator 120, the blood temperature may be increased through heat exchanger 122 (FIG. 1a) to a desired level. Oxygenator 120 is necessary in cases where complete isolation of coronary circulation is desired.

Enricher 130 allows blood, e.g. in which oxygenated and detoxified blood, to be further processed. Processing in Enricher 130 may include including enrichment of blood with needed nutrients, drugs, including any high dosage level drugs, or other desired substances, e.g. those needed to stabilize an atherosclerotic plaque. Examples of substances needed to stabilize an atherosclerotic plaque and other therapeutic agents may include HDL or its main apoprotein (apoA-1), statins, chelating agents, genes, hyperbaric oxygen in case of acute coronary syndrome, or the like, or combinations thereof. Blood may also be enriched by adding therapeutic agents such as HDL, chelating agents, transgenes, and any drugs such as statin that may be delivered directly into the coronary circulation.

Heater 140 may be present to heat blood to a desired temperature, e.g. a therapeutic temperature of around 41-42° C.

Pump 150 allows fully processed blood to be pumped back to patient 10 with appropriate volume, perfusion pressure (flow), and appropriate temperature back into the isolated coronary arterial system of patient 10.

In a preferred embodiment, chambers 110-150 are arranged in series with a predetermined sequencing. For example, chambers 110-150 may be configured to promote ameliorating of the blood sequentially, e.g. first cleaning harmful/unwanted materials from blood, then oxygenate the blood, then warm the blood, then enrich the blood with nutrients or drugs, and finally pump the blood back to the coronary system in this order to deliver, in a continuous fashion, a dose, including a high dose, of drugs in direct vicinity of coronary arteries and their endothelium an well as sub-endothelial layers.

Whether all or some of these chambers 110-150 are used may depend on the design of perfusing catheter 220 (FIG. 2) and collecting catheter 210 (FIG. 5a) used in a particular method. When complete isolation of coronary circulation is desired, it may be necessary to include blood separation chamber 110, oxygenator 120, enrichment chamber 130, blood heating chamber 140, and blood pump chamber 150 in multi-chambered dialysis machine 100. In cases where complete isolation of coronary circulation is not established, oxygenator 120 may not be needed if sufficient arterial oxygenated blood flows into the coronary artery. Therefore only blood separation chamber 10, enrichment chamber 120, blood heating chamber 140, and blood pump chamber 150 may be sufficient for blood processing and delivery in these latter cases.

In an embodiment, multi-chambered coronary dialysis machine 100 may comprise one or more microprocessors or other controllers (not shown in the figures) to aid in automatically providing for separation, treatment, and dialysis of blood with monitoring of the extracorporeal plasma circuit. Multi-chambered dialysis machine 100 may also be added to a hemodialysis machine to be used in patients suffering from chronic renal failure who are at high risk for atherosclerosis and its complications, e.g. diabetes mellitus patients.

Referring now to FIG. 2, catheter system 200 (FIG. 1) comprises a novel catheter system for either complete or incomplete isolation of the coronary circulatory system of patient 10 (FIG. 1) from systemic circulation, e.g. by means of ante grade perfusion catheter 220 introduced percutaneously from out of the body. Catheter system 200 acts as a delivery system for local introduction of processed blood directly into the coronary arteries.

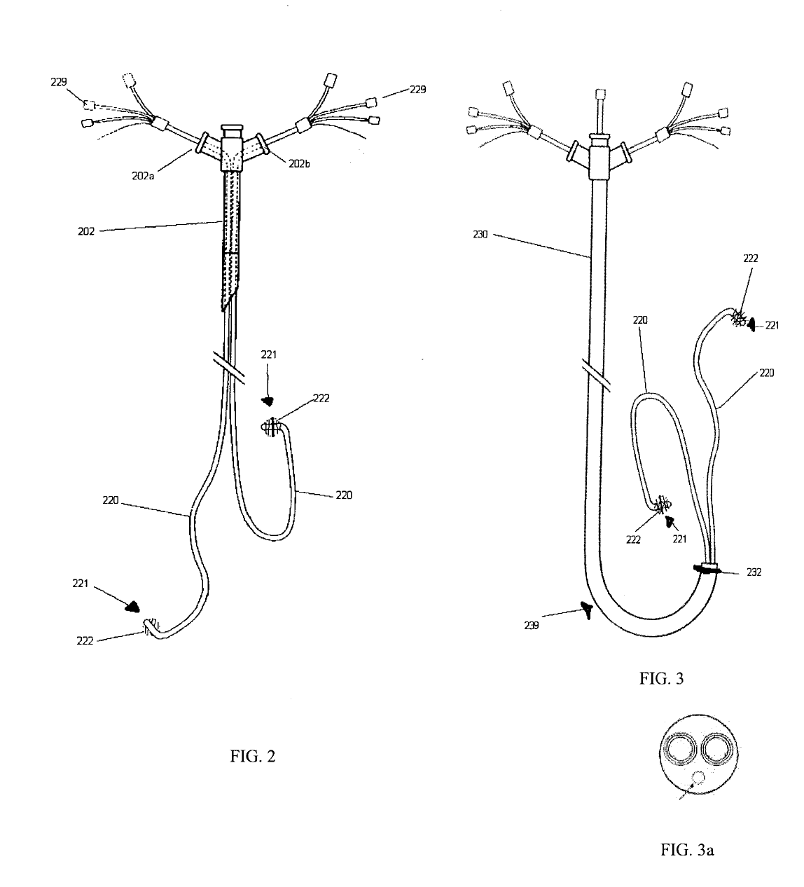

Catheter system 200 (FIG. 1) may comprise several configurations designed to introduce processed blood by direct ante grade perfusion into the coronary arteries. For example, configurations may comprise presence or absence of vehicle catheter 230 (FIG. 3) for ante grade perfusion, presence or absence of inflatable balloon 232 (FIG. 3) at distal tip 231 (FIG. 3) of vehicle catheter 230 or inflatable balloon 222 at distal tip 221 of perfusion catheter 220, and/or variation of inflatable balloon 222 for complete or incomplete isolation of the coronary circulation.

The coronary ostia may be occluded to substantially completely isolate coronary arteries from systemic circulation in order to prevent dilution of processed blood by the systemic blood. Consequently, high level of drug or therapeutic agent may be delivered to the intimate vicinity of coronary artery endothelial cells.

Alternatively, the coronary ostia may be left non-occluded so that the coronary artery system is perfused with processed blood as well as blood from the systemic circulation. By exposing endothelial cells to high level of local HDL, statin, genes, or chelating agents, it is anticipated that the present system would enhance the physiologic effects of these delivered therapeutic agents in lipid metabolism as well as plaque composition. As a result, lipid laden, high macrophage and low smooth muscle cell containing vulnerable plaques would undergo constitutional changes by decreasing lipid as well as macrophage content. When the plaques are stabilized in this way, plaque rupture and its consequences may be lessened if not prevented.

Referring now to FIG. 5a, catheter system 200 may comprise arterial introducer 202, collection catheter 210, perfusion catheter 220, and vehicle catheter 230. As used herein, perfusion catheter 220 is equivalent to a perfusion dialysis catheter.

Arterial introducer 202 may be introduced through femoral artery 30. Collecting catheter 210 may be introduced into coronary sinus ostium 27 (FIG. 5c) and completely occlude the ostium to collect and drain blood out of patient 10 for processing. Collecting catheter 210 may comprise multiple configurations, e.g. variations in catheter physical designation as well as differing configurations of balloon 212 (not shown in the figures).

Referring back to FIG. 2, FIG. 2 illustrates the design of a perfusion catheter 220. Arterial sheath 202 is introduced through the femoral artery and comprises two or more ports through which one or more perfusion catheters 220 may be introduced and passed up to the coronary artery ostia. The distal end of perfusion catheter 220 has pre-shaped curvature so that it may be engaged into the coronary artery ostia readily. There is a lumen extending throughout either the entire length or a portion of perfusion catheter 220 through which the processed blood may be perfused to the coronary circulation. The lumen of perfusion catheter 220 is connected to the outlet of the dialysis machine. An inflation channel may also exist within or proximate the wall of perfusion catheter 220 for balloon inflation.

Sensors may be present within the lumen of perfusion catheter 220, e.g. for detecting the temperature as well as perfusion pressure of the blood. The sensors are connected via a wired or wireless method to monitoring system 300. In a preferred embodiment, separate wires are used to connect each sensor to monitoring system 300.

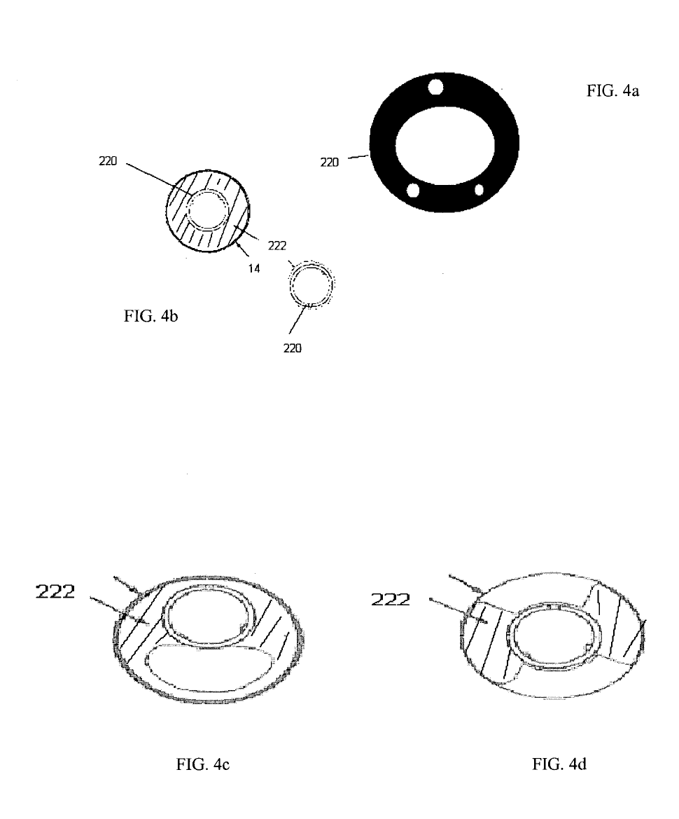

Tip 221 of perfusing catheter 220 may be equipped with inflatable balloon 222, as illustrated in FIG. 1, which may occlude coronary ostium completely. Alternatively, tip 221 of perfusing catheter 220 may be equipped with inflatable ring shape balloon, as illustrated in FIG. 2, or an inflatable butterfly shape balloon as illustrated in FIG. 3, that does not occlude the coronary ostium completely. FIG. 4 illustrates tip 221 of perfusing catheter 220 without inflatable balloon 222.

In another embodiment, tip 221 of perfusing catheter 20 may have a specific space around tip 221, e.g. annulus 221a, operatively connected to a vacuum system (not shown in the figures). Upon engaging a portion of tip 221 into the coronary ostium, e.g. the first 2-3 mm, a negative pressure may be generated within this space to facilitate the attachment and fixation of perfusion catheter 220 in the coronary ostium. In such an embodiment, attaching perfusion catheter 220 to the aortic wall adjacent to the coronary ostium by vacuum eliminates the need for any inflatable balloon 222.

Arterial introducer 202 may comprise ports 202a, 202b through which catheters, e.g. two perfusion catheters 220, may be introduced and passed up to the coronary artery ostia. Ports 202a, 202b may also provide entry and exit points for channels and wires present with the catheters.

In a preferred embodiment, perfusion catheter 220 is substantially tubular with an outer wall defining an interior lumen. Distal end 221 of perfusion catheter 220 may comprise a pre-shaped curvature so that it may be engaged into the coronary artery ostia more readily. Perfusion catheter 220 may have balloon 222 at tip 221.

As perfusion catheter may comprise a tubular portion, one or more lumen may extend throughout the length of perfusion catheter 220 to allow processed blood to be perfused to the coronary circulation. The lumen of perfusion catheter 220 may be adapted to connect to fluid outlet 104 (FIG. 1a) of multi-chambered dialysis machine 100. As illustrated in FIG. 3a, inflation channel 227 may be present within or proximate to the outer wall of perfusion catheter 220 for inflation of balloon 222.

One or more sensors may be disposed proximate or within the lumen of perfusion catheter 220 such as for detecting blood temperature, perfusion pressure of the blood, or the like, or a combination thereof. Sensors may be connected to monitoring system 300, e.g. using wired or wireless connections, for detection and/or monitoring of blood temperature and pressure respectively.

The lumen of perfusing catheters 220 should have sufficient internal diameter to allow a flow rate of about at least 150 ml/min for processed blood, with a preferred range being 150-250 ml/min. Further, perfusing catheters 220 should be able to maintain a safe coronary perfusion pressure of about 100-150 mmHg in case of complete isolation of coronary artery.

Perfusion catheter 220 may further contain inflation channel 238 (FIG. 4a) proximate the outer wall. Inflation channel 238 may be in fluid communication with and help inflate inflatable balloon 222.

In one embodiment, inflatable balloon 222 may occlude the coronary ostium completely when inflatable balloon 222 is inflated (FIG. 4b). Alternatively, inflatable balloon 222 may be ring-shaped (FIG. 4c) or butterfly-shaped (FIG. 4d) so that inflatable balloon 222 cannot occlude the coronary ostium completely when it is inflated.

One or more lumen may extend throughout the length of perfusion catheter 220 through which the processed blood may be perfused to the coronary circulation. Lumen of perfusion catheter 220 may be fluidly connected to fluid outlet 104 (FIG. 1a) of multi-chambered dialysis machine 100.

In an embodiment, two sensors are disposed proximate or within lumen of perfusion catheter 220 for detecting temperature as well as perfusion pressure of the blood. The sensors may be connected via two separate wires to monitoring system 300 for detection of blood temperature and pressure respectively.

Referring now to FIG. 3, an illustration of vehicle catheter 230 with aortic root ring 232, vehicle catheter 230 may be introduced through femoral artery 30 (FIG. 5a) of patient 10 and may comprise pre-shaped curvature 239 proximate tip 231 where pre-shaped curvature 239 may be compatible with aortic arch 22 (FIG. 5a). Throughout the length of vehicle catheter 230, one or more channels, e.g. 233-234 (FIG. 3a), may be present, e.g. channel 233 for monitoring the blood pressure within the ascending aorta, inflation channel 234 disposed proximate wall 235, and the like.

Inflatable aortic root ring 232 may be present at tip 231 and may be in fluid communication with inflation channel 234 for inflating and/or deflating inflatable aortic root ring 232. Inflatable aortic root ring 232 may be used to help stabilize vehicle catheter 230 in aortic arch 22 when inflatable aortic root ring 232 is inflated.

In an embodiment, two or more perfusion catheters 220 may be contained at least partially or otherwise housed within vehicle catheter 230.

FIG. 5 illustrates vehicle catheter 230 without aortic root ring 232. A single vehicle catheter 230 with a pre-formed curvature at tip 231 compatible with the aortic arch may be introduced through femoral artery 30 up to the ascending aorta above the coronary ostia. Channel 233 may extend throughout the length of vehicle catheter 230 where channel 233 is adapted for use in monitoring the blood pressure within the ascending aorta. Two or more perfusion catheters 220 may be at least partially contained or housed within lumen of vehicle catheter 230.

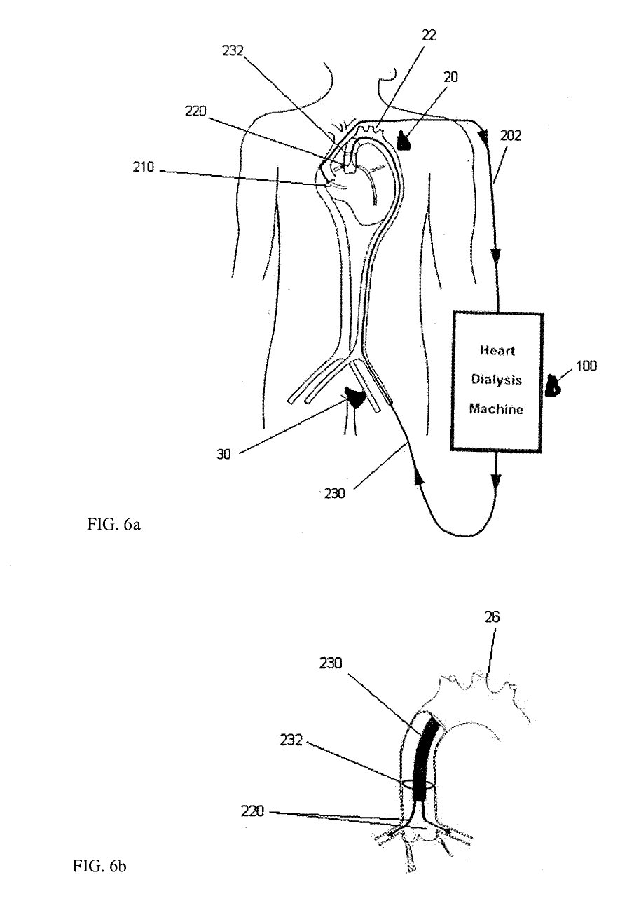

Vehicle catheter 230 may be equipped with aortic root ring 232. In an embodiment, after perfusion catheter 220 housed within vehicle catheter 230 is engaged into the coronary ostium, ring shape balloon 232 may be inflated, e.g. using inflation channel 234, to stabilize vehicle catheter 230 within the aortic root (FIG. 6C).

In the operation of several exemplary embodiments, coronary dialysis system 1 and its methods of use comprise a capability to be used not only for patient 10 already suffering from chronic coronary artery disease but also its usage in the setting of acute coronary syndrome (“ACS”), e.g. unstable angina and acute myocardial infarction, to decrease plasma factors which affecting the short and long term survival of patients. Coronary dialysis system 1 and/or multi-chambered dialysis machine 100 may be used with patients 10 known to be suffering from coronary artery disease in a stable clinical situation as well as implemented in an ACS setting in order to reduce the plasma level of harmful and toxic factors such as CRP, tissue factor as well as fibrinogen released during acute coronary syndrome. Consequently, the complication, morbidity and mortality of acute coronary syndrome may be reduced.

Coronary dialysis system 1 may be used to deliver ante grade, local, direct, and high dose of therapeutic agents such as HDL, genes, chelating agents, and statin to coronary arterial system. A high level of therapeutic agents may be maintained in the intimate vicinity of coronary endothelial cells by preventing dilution of processed blood by the systemic circulation. Hence, maximal therapeutic effects may be obtained.

Accordingly, one or more methods of using coronary dialysis system 1 comprises use of multi-chambered coronary dialysis machine 100 to perform one or more specific functions, which can be accomplished in a specific, e.g. serial, order to reduce risk factors of atherosclerosis, prevent the progression of atherosclerosis, and/or prevent the occurrence of acute and chronic complications of atherosclerosis such as acute coronary syndrome and congestive heart failure. These methods may be used to deliver a number of therapeutic agents locally into the coronary system. Representative therapeutic agents may include, but are not limited to, statins, anti-inflammatory agent, angiotensin converting enzyme inhibitor, peroxisome proliferator-activated receptor agonist, HDL, apolipoprotein apoA1, mutated apolipoprotein apoA1, gene for gene therapy and chelating agent. Further, harmful and/or unwanted substances may be removed from the blood. Examples of these substances include cholesterol, LDL, triglyceride, perfused HDL, C-reactive protein, Lp (a), fibrinogen, tissue factor, interleukine, interleukine 1, interleukine 6, TNF-alpha, chemoattractant molecules, CD 14, C3 complement, C4 complement, and C 1 inhibitor.

Moreover, presence of an inappropriate high plasma level of drugs or any added agents may be prevented by plasmaphaeresis as well as aphaeresis in multi-chambered dialysis machine 100. Consequently, the coronary circulation may be perfused with appropriate level of drugs and other substances that make the plaque stable (e.g. HDL may mobilize cholesterol from peripheral tissue through reverse cholesterol transport), and harmful and/or unwanted substances such as LDL may be removed from the processed blood through processing (e.g. aphaeresis) in multi-chambered dialysis machine 100.

Coronary dialysis system 1 may be used to access the circulatory system from a peripheral venous site or a peripheral induced shunt in order to establish rapid and aggressive therapy for treatment of patient and his coronary vulnerable plaques. Coronary dialysis system 1 may further be used to provide continuous delivery of high doses of affecting drugs (such as cholesterol removal drugs, anti inflammatory, anti thrombotic, thrombolytic therapy, and/or chelating agents, or the like, or combinations thereof), and genes for treatment of cardiovascular diseases such as coronary atherosclerosis and acute coronary syndrome. Coronary dialysis system 1 allows delivery of dosages, including high dosages, of substances such as HDL in order to facilitate, enforce, and potentates their effect on atherosclerotic plaque. For example, during high dose delivery of chelating agents and genes other therapeutic modalities may be followed.

Referring now to FIG. 5a, in several preferred embodiments, coronary dialysis system 1 is configured as a substantially closed fluid circuit in which the cardiac circulatory system of patient 10 may be completely isolated from systemic circulation. In this embodiment, collecting catheter 210, perfusing catheter 220, multi-chambered dialysis machine 100, and the circulatory system of patient 10—e.g. the coronary arterial tree, cardiac capillary system, and cardiac venous system of patient 10—are incorporated in series in a loop circuit.

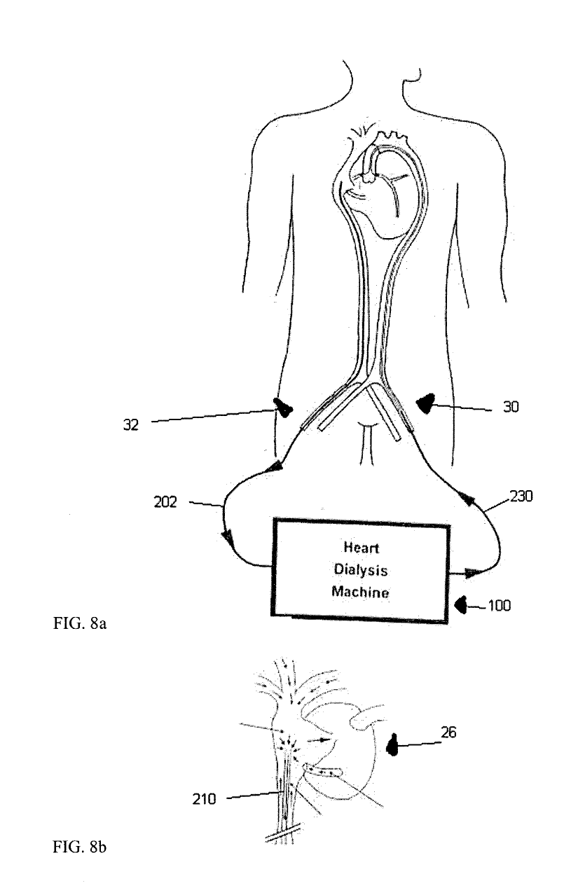

Collecting catheter 210 may be introduced at a left subclavian vein, right subclavian vein, right jugular vein, and/or femoral vessels 30,32 (FIG. 8).

Referring now to FIG. 6a, in one such embodiment, after introducing vehicle catheter 230 through arterial sheath 202 and inflating ring shaped balloon 232 to fix vehicle catheter 230 in the ascending aorta, one or more perfusing catheters 220 specifically designed for individual coronary ostium are introduced and engaged appropriately in each desired coronary artery, e.g. through femoral artery 30. Ostia may be occluded by inflating balloons 222 (FIG. 2) at tips 221 (FIG. 2) of perfusing catheters 220. If inflatable balloon 212 is present, it may either substantially occlude or partially occlude coronary sinus 27. For example, at the end of the cardiac venous drainage system, e.g. at coronary sinus 27 (FIG. 5c), collecting catheter 210 with inflatable balloon 212 (not shown in the figures) at tip 211 (not shown in the figures) is engaged into coronary sinus 27 where inflatable balloon 212 is inflated. If inflatable balloon 212 is ring shaped, occlusion may occur. If inflatable balloon 212 is butterfly shaped, occlusion may not occur.

The venous blood of heart 20 is drained to multi-chambered dialysis machine 100. Processed blood is returned to patient 10 via one or more perfusion catheters 220 into one or more blood vessels (FIG. 5b).

In general, these methods may provide treatment such as stabilizing vulnerable plaques, decreasing the lipid content of the plaques, reducing inflammatory activity throughout the coronary system, decreasing the number of macrophages in the plaques and increasing the number of smooth muscle cells in the plaques. A number of coronary diseases such as atherosclerosis, chronic coronary artery disease, acute coronary syndrome, unstable angina, and acute myocardial infarction may be treated by these methods.

Using coronary dialysis system 1, blood may be processed using plasmaphaeresis (primary plasma separation) and aphaeresis (plasma secondary separation). In general, based on plasmaphaeresis and aphaeresis, multi-chambered dialysis machine 100 may reduce harmful substances from blood through the processes of precipitation, filtration, adsorption, immune precipitation, immunoabsorption, or the like, or a combination thereof, e.g. by using micro-beads or nanoparticles.

After blood is withdrawn from patient 10, as in the cases of conventional hemodyalysis in patients 10 suffering from renal failure, the blood is drained to multi-chambered dialysis machine 100 via fluid inlet 102 (FIG. 1a) to start processing in blood separation chamber 110 (FIG. 1a). By plasmapheresis, blood cells may be separated from plasma and the plasma may undergo a further step, e.g. apheresis. Using these steps, harmful and unwanted plasma substances may be separated from the plasma, e.g. LDL, triglyceride, CRP, Lp (a), fibrinogen, tissue factor, and several plasma cytokines such as interleukine 1 and 6, TNF-alpha, adhesion molecules (ICAM-1, VCAM-1), chemoattractant molecules such as monocyte colony stimulating factor, CD14, as well as 0 complement, C4 complement, and/or C1 inhibitor, or the like, or combinations thereof.

In an embodiment, plasma differential precipitation is applied for the elimination of LDL-cholesterol. After being separated from a hollow fiber module plasma will be acidified and heparinized. The precipitated plasma components are separated from a second hollow fiber module. The heparin excess is removed from the adsorption column from the plasma. The naturalized plasma after dialysis and ultra filtration will be re-admixed with blood cells. By implementing this method not only LDL but also a lot of the plasma substances will be co-precipitated and separated from plasma such as CRP, fibrinogen, tissue factor, CD14, LP (a), inter leukin-1, TNF alpha, inter leukin-6, C3, C4, C1 inhibitor, ferittin, and/or plasminogen, or the like, or combinations thereof.

Immunoabsorption (immunoprecipitation) using different ligands such as amino acids, protein, and polyclonal antibody is the mechanism that may be used to selectively precipitating and separating the plasma harmful materials including the factors mentioned above or even more other harmful factors.

In a preferred embodiment, blood may be warmed to an appropriate temperature, e.g. around 41-42° C. Heating the blood may not only directly subside inflammatory process but also, through melting and de-crystallization of cholesterol, may help plaque stabilization. Furthermore, blood heating to the febrile range has systemic effect that may decrease the plasma level of some cytokines such as TNF-alpha, IL-1, and IL-6. This heating process may be done in heater chamber 140 and/or in heat exchanger 122.

Blood may be passed to Enricher 130 (FIG. 1a) in which dosages, including high dosages, of drugs and/or other therapeutic agents may be added to the passing blood to be delivered to the systemic circulation as well as coronary circulation in order to prevent atherosclerotic plaque progression and provide plaque stabilization. HDL, and its main apoprotein apoA-1, is the mainstay agent that may be perfused systemically.

Other classes of agents that could be utilized and perfused systemically may comprise genes such as gene encoding the apoA-1 in the liver to increase its production. Gene transfer to stabilize the vulnerable atherosclerotic plaque may prevent plaque rupture and subsequent thrombosis. Possible strategies include over-expressing TIMPs (tissue inhibitor of matrix metalloproteinase) and blocking the actions of pro-inflammatory molecules such as those of the transcription factor NF-κB.

Other agents that may be perfused comprise chelating agents such as EDTA to extract the calcium content of the atherosclerotic plaques throughout of the circulatory system of the body including coronary arteries.

Using this system which provides the procedure for specially removing APO-containing ffpoproteins from the body. This technique is based upon the precipitation of the positively charged LDL and other beta lipoprotein when heparin is added at low PH. In addition of LDL a number of other plasma proteins such as LP (a), fibrinogen, plasminogen, antithrombin, TNF alpha, CD14, CD40/CD40L, circulating adhesion molecules, and C3 and c4 are all co-precipitated.

Dyslipidemias remain important target for the development of novel therapies. Gene therapy is a logical therapeutic approach to monogenic lipoprotein disorder, such as homozygous familial hypercholesterolemia, familial lipoprotein lipase deficiency, familial LCAT deficiency, and abetalipoproteinemia for which current therapies are inadequate.

Gene therapy could theoretically stabilize the vulnerable plaque by reducing the plaque content in lipids and macrophages. Alternatively, the introduction into the atherosclerotic plaque of genes encoding for thrombolytic proteins or growth factors able to restore physiologic antithrombotic function of endothelial cells may inhibit thrombus formation should the plaque rupture. Although many technical challenges still lie ahead, recent developments indicate they are possibly within reach in the nottoo distant future.

Gene therapy could also be used to increase the expression of certain protein, such as apo a-1 as strategy to raise HDL cholesterol level or apo E as a strategy for severe combined hyper lipidemia. With further progress in development of vectors, gene therapy for severe dyslipidemia likely to become a clinical reality.

Liver directed gene transfer of human apoA-1 resulted in significant regression of the preexisting atherosclerotic lesion in LDL receptor deficient mice as assessed by important methods. Apo a-1 and LCAT are two potential targets for gene therapy in patients with atherosclerosis associated with a low HDL cholesterol level.

FGF-4(GENEREX) is a angiogenic gene therapy which triggers the production of a protein that stimulate new blood vessel growth providing an attractive route for blood to by pass clogged and blocked arteries in the heart. GENEREX in an one- time non surgical delivery of an adenovirus vector containing the human FGF-4 in to the coronary arteries via a standard catheter.

PhVEGF-A165 injection directly in to myocardium at four sites in the anterolateral region of left ventricle was done. Plasma VEGF-165 increased peaking at sixth day. It has significant effect in decreasing angina pectoris, nitroglycerine intake and improving CCS. These improvements remained after 12 months. So intramyocardial injection of phVEGF-165 is safe and may lead to improved myocardial perfusion and function with longstanding symptomatic relief at end stage angina pectoris.

Chelation is the binding (and subsequent elimination) of harmful substances that are present in the bloodstream and in the walls of hardened and partially clogged blood vessels.

“EDTA” is a meta 1-complexing synthetic amino acid that acts as the “chelator.” “EDTA” chelation is a therapy by which repeated administrations of a weak synthetic amino acid (“EDTA,” ethylenediamine tetra-acetic acid) gradually reduce atherosclerotic plaque and other mineral deposits throughout the cardiovascular system by literally dissolving them away. “EDTA” infusion removes the calcium which is necessary for the formation of fibrinogen and coagulation from the blood stream.

The following exemplary method embodiments are given for the purpose of illustrating various embodiments of the invention and are not meant to limit the present invention in any fashion.

Referring still to FIG. 5a, in a first method embodiment, arterial sheath 202 is introduced, such as by using Seldinger's method, after obtaining access site in femoral artery 30. Two perfusion catheters 220, one for each of two coronary arteries (FIG. 5b), may be introduced through vehicle catheter 230. Distal end 221 of perfusion catheter 220 may have a pre-shaped curvature so that distal end 221 of perfusion catheter 220 may be engaged into the coronary artery ostia more readily. In a first exemplary method embodiment, there is no occluding balloon 222 so that the heart is not isolated from systemic circulation.

Collecting catheter 210 may be introduced, such as through the right subclavian vein or right jugular vein directly into right atrium 24, to collect the venous blood and drain it to multi-chambered dialysis machine 100 for processing.

Alternatively, inflatable balloons 212 and/or 222 may be present but may comprise a ring or butterfly shape on inflation that do not completely block the coronary ostia.

In this exemplary method embodiment, the re-circulating blood does not need to be oxygenated and coronary perfusion pressure is regulated by systemic blood flow. Processed blood, however, may be diluted by the systemic blood.

Referring now to FIG. 6a, in a second method embodiment, arterial sheath 202 is introduced, such as by using Seldinger's method, after obtaining access site in femoral artery 30. Two perfusion 220, one for each of the two coronary arteries, may be introduced through arterial sheath 202.

Collecting catheter 210 may be introduced, such as through the right subclavian vein or right jugular vein directly into right atrium 24, to collect the venous blood and drain it to multi-chambered dialysis machine 100 for processing.

These catheters 210 and 220 have inflatable balloons at their tips, i.e. inflatable balloon 212 at tip 211 and inflatable balloon 222 at tip 221. Inflatable balloons 212,222 may completely block the coronary ostia to isolate the coronary system from systemic circulation. In this exemplary method, processed blood is perfused directly to the coronary artery without dilution while maintaining a dose of drugs in direct contact to endothelial cells, including high doses of drugs. In this method, multi-chambered dialysis machine 100 further comprises oxygenator 120 (FIG. 1a) as well as pump 150 (FIG. 1a).

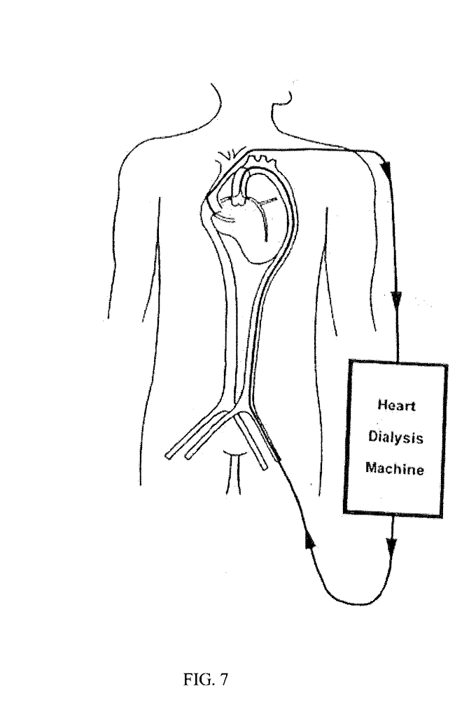

Referring now to FIG. 7, in a exemplary third embodiment, arterial sheath 202 is introduced, such as by using Seldinger's method, after obtaining access site in the femoral artery. Then a single catheter, vehicle catheter 230 with a pre-formed curvature at distal 231 which is compatible with aortic arch 22, is introduced through arterial sheath 202 up to the ascending aorta above coronary ostia. Vehicle catheter 230 may or may not have an inflatable ring-shape balloon 232 at tip 231 for fixation and stabilization of vehicle catheter 230 in the ascending aorta.

Collecting catheter 210 is introduced and takes the venous blood out of patient 10 to multi-chambered dialysis machine 100 for processing. Collecting catheter 210 may be applied by introducing a pre-shaped collecting catheter 210, such as through the left subclavian vein using Seldinger's method, to engage into coronary sinus 27. After engaging collecting catheter 210 in the coronary sinus ostium, inflatable balloon 212 is inflated and coronary sinus 27 will be occluded. Collecting catheter 210 may then collect the venous blood of the heart and drain to multi-chambered dialysis machine 100.

Currently contemplated variants of this embodiment are related coronary perfusing catheters 220 which may or may not have inflatable balloons 222 at their tips 221. As discussed herein above, perfusing catheter 220 may comprise inflatable balloon 222 which completely or substantially completely occludes coronary artery ostia so that complete isolation of heart 20 may be achieved. Preferably the lengths of perfusing catheters 220 allow perfusing catheters 220 to be introduced via femoral artery 30 to the coronary ostia. The lengths of vehicle catheter 230 similarly should allow perfusing catheters 220 to be introduced via femoral artery 30 to aortic root 22.

Distal end 221 of each perfusing catheter 220 may be preshaped with an appropriate curvature and angulations similar to those in standard coronary angiography catheter so that perfusing catheter 220 may be selectively introduced into each coronary ostium as coronary catheters. Alternatively, perfusing catheter 220 may be so designed that the curvature and angulations of tip 221 may be changed by implementing fibers along the length of perfusing catheter 220 to facilitate coronary engagement.

A fourth exemplary embodiment is similar to the third exemplary embodiment except perfusing catheter 220 either does not comprise balloon 222 or may comprise a non-occluding balloon 222. Where perfusing catheter 220 does not comprise balloon 222 or comprises a non-occluding balloon 222, dilution of processed blood by systemic blood and instability of catheters 220 at coronary ostia may be of concern. However, co-perfusion of the coronary artery by systemic circulation omits the necessity of providing oxygenator 120 (FIG. 1a).

Referring now to FIG. 8, a fifth exemplary embodiment is similar to the exemplary embodiments above except that perfusion catheter and collection catheters are introduced through femoral vessels, e.g. 30 and 32, rather than using a subclavian vein.

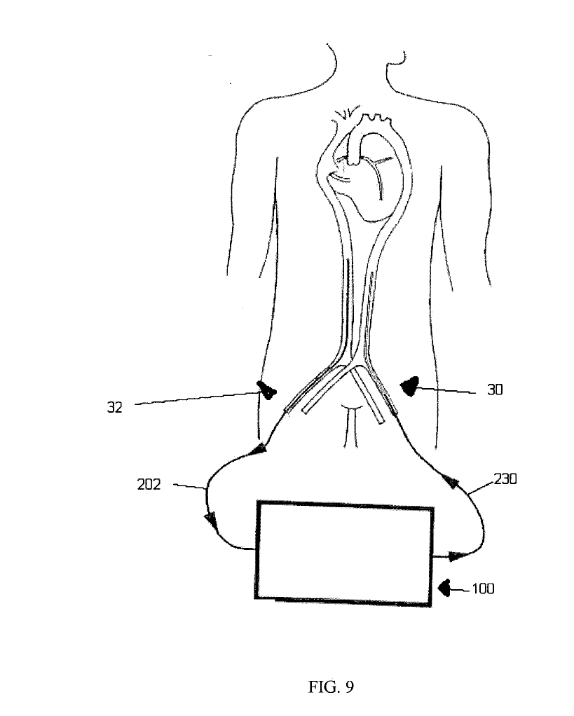

Referring now to FIG. 9, in a sixth exemplary embodiment systemic dialysis may be obtained by fitting patient 10 with collection catheter 210 and perfusion catheter 220, e.g. using femoral artery 30 and femoral vein 32. In this embodiment, oxygenator 120 may not be required. Further, use of inflatable balloons 222,232 may not be required.

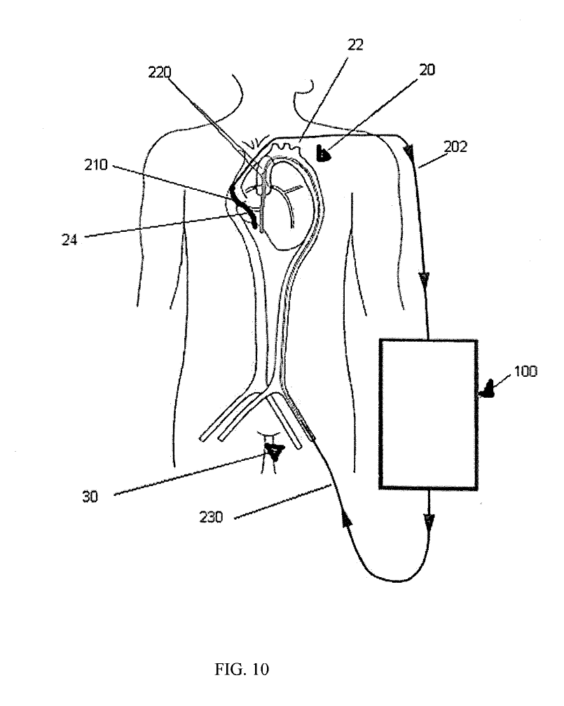

Referring now to FIG. 10, in a seventh exemplary embodiment, fluids within the pericardium, as well as external heart tissue, may be remediated by introducing collection catheter 210 and perfusion catheter 220 into the space intermediate the pericardium and the heart. As with the methods described herein above, fluid may be withdrawn, processed, and returned to the space intermediate the pericardium and the heart. In an embodiment, entry into the pericardium may be accomplished via the aorta.

It will be understood that various changes in the details, materials, and arrangements of the parts which have been described and illustrated above in order to explain the nature of this invention may be made by those skilled in the art without departing from the principle and scope of the invention as recited in the appended claims.

What is claimed is:

1. A coronary dialysis system, for a patient comprising a coronary arterial tree, a cardiac venous system, and a cardiac capillary system, the coronary dialysis system comprising:
a. a multi-chambered dialysis machine, comprising a fluid inlet in fluid communication with a fluid outlet, the multi-chambered dialysis machine adapted to separate plasma from blood and process the blood to at least one of (i) remove an unwanted substance from the blood or (ii) add a desired substance to the blood;
b. a collecting catheter system in fluid communication with the patient and the fluid inlet; and
c. a coronary artery perfusing catheter in fluid communication with the patient and the fluid outlet.

2. The coronary dialysis system of claim 1, further comprising at least one of (i) an arterial sheath adapted to receive a plurality of catheters therethrough or (ii) a vehicle catheter adapted to pass through the arterial sheath, the vehicle catheter adapted to receive a plurality of catheters therethrough.

3. The coronary dialysis system of claim 1, wherein the multi-chambered dialysis machine further comprises:
a. a blood separation chamber in fluid communication with the input and output; and
b. a blood processing chamber in fluid communication with the input, the output, and the blood separation chamber.

4. The coronary dialysis system of claim 3, wherein the blood separation chamber further comprises:
a. a first hollow fiber module adapted to provide plasma differential precipitation;
b. an acidifier in fluid communication with the first hollow fiber module;
c. a heparinizer in fluid communication with the acidifier;
d. a second hollow fiber module in fluid communication with the heparinizer; and
e. an adsorption column in fluid communication with the second hollow fiber module.

5. The coronary dialysis system of claim 3, wherein the blood separation chamber is adapted to accomplish at least one of (i) immune precipitation or (ii) immunoabsorption using at least one of (iii) micro beads or (iv) a nanoparticle.

6. The coronary dialysis system of claim 3, wherein:
a. the blood separation chamber further comprises at least one of (i) a plasmaphaeresis chamber or (ii) an aphaeresis chamber; and
b. the blood processing chamber further comprises at least one of (i) an oxygenator, (ii) an enricher, (iii) a heater, or (iv) a pump.

7. The coronary dialysis system of claim 6, wherein the heater is adapted to warm blood to between around 35° C. to around 45° C.

8. The coronary dialysis system of claim 6, wherein the enricher is adapted to provide at least one of (i) a predetermined dose of a drug or (ii) a therapeutic agent.

9. The coronary dialysis system of claim 2, wherein a length of the arterial sheath is sufficient to allow a catheter disposed within the arterial sheath to be introduced via a femoral artery to an aortic root.

10. The coronary dialysis system of claim 2, wherein the vehicle catheter further comprises an inflatable balloon disposed proximate a distal end of the vehicle catheter, the inflatable balloon adapted to engage against the ascending aorta.

11. The coronary dialysis system of claim 1, wherein a length of a catheter is sufficient to allow the catheter to be introduced via a femoral artery to a coronary ostium, the catheter comprising at least one of (i) the coronary perfusing catheter or (ii) the perfusion catheter.

12. The coronary dialysis system of claim 1, wherein the perfusion catheter further comprises a tubular portion, the tubular portion adapted to provide at least one of (i) a sufficient internal diameter to allow a flow rate of about 150-250 ml/min or greater for processed blood or (ii) coronary perfusion pressure of about 100-150 mmHg.

13. The coronary dialysis system of claim 1, wherein a distal end of the perfusion catheter comprises a pre-shaped curvature adapted to allow engagement of the distal end of the perfusion catheter into a coronary artery ostium.

14. The coronary dialysis system of claim 13, wherein the pre-shaped curvature is adapted to be compatible with an aortic root.

15. The coronary dialysis system of claim 1, wherein each of the collecting catheter and the perfusion catheter further comprise an inflatable balloon disposed proximate a distal end of that catheter.

16. The coronary dialysis system of claim 15, wherein the inflatable balloon is adapted to at least one of (i) substantially completely block a coronary ostium to isolate the coronary system from systemic circulation or (ii) not completely block the coronary ostium.

17. The coronary dialysis system of claim 16, wherein, upon inflation, the inflatable balloon adapted to not completely block the coronary ostium comprises at least one of (i) a ring shape or (ii) a butterfly shape.

18. The coronary dialysis system of claim 1, wherein the perfusing collecting catheter further comprises:
a. a changeable curvature; and
b. a changeable tip angulation.

19. The coronary dialysis system of claim 18, wherein curvature and tip angulation may be changed implementing fibers along the length of the catheter to facilitate coronary engagement.

20. The coronary dialysis system of claim 1, wherein:
a. the collecting catheter is adapted to be engaged in the coronary sinus ostium to collect venous blood from the patient; and
b. the perfusion catheter is adapted to be engaged into the coronary artery ostia of the patient.

21. The coronary dialysis system of claim 11, wherein the perfusion catheter further comprises a sensor.

22. The coronary dialysis system of claim 21, wherein the sensor further comprises:
a. a temperature sensor disposed proximate a wall of the tubular component; and
b. a pressure sensor disposed proximate the wall of the tubular component.

23. The coronary dialysis system of claim 22, further comprising:
a. a monitoring system for detection of blood temperature and pressure respectively, the monitoring system operatively in communication with at least one of (i) the temperature sensor or (ii) the pressure sensor.

24. The coronary dialysis system of claim 12, wherein the perfusion catheter further comprises an inflation channel disposed proximate a wall of the tubular component.

25. The coronary dialysis system of claim 11, wherein:
a. the vehicle catheter further comprises a pressure channel adapted to monitor blood pressure within the ascending aorta; and
b. the perfusion catheter comprises a plurality of perfusion catheters contained at least partially within the vehicle catheter.
Catheter Config

26. A catheter system for coronary dialysis, comprising: %
a. a collecting catheter, comprising:
i. an inlet adapted to receive blood from a coronary site in a patient; and
ii. an outlet, adapted to deliver blood to a dialysis machine; and
b. a coronary artery perfusing catheter; comprising:
i. an inlet adapted to receive blood from the dialysis machine; and
ii. an outlet, adapted to deliver blood to a coronary site in a patient.

27. The catheter system of claim 26, wherein each of the coronary perfusing catheter and the perfusion catheter catheter is sufficient to allow the catheter to be introduced via a femoral artery to a coronary ostium.

28. The catheter system of claim 26, wherein the perfusion catheter further comprises a tubular portion, the tubular portion adapted to provide at least one of (i) a sufficient internal diameter to allow a flow rate of about 150-250 ml/min or greater for processed blood or (ii) coronary perfusion pressure of about 100-150 mmHg.

29. The catheter system of claim 26, wherein a distal end of the perfusion catheter comprises a pre-shaped curvature adapted to allow engagement of the distal end of the perfusion catheter into a coronary artery ostium.

30. The catheter system of claim 29, wherein the pre-shaped curvature is adapted to be compatible with an aortic root.

31. The catheter system of claim 26, wherein the catheter system further comprises an arterial sheath adapted to received at least one of (i) the collecting catheter or (ii) the perfusion catheter.

32. The catheter system of claim 31, wherein a length of the arterial sheath is sufficient to allow a catheter disposed within the arterial sheath to be introduced via a femoral artery to an aortic root.

33. The catheter system of claim 31, further comprising a vehicle catheter adapted to pass through the arterial sheath.

34. The catheter system of claim 33, wherein the vehicle catheter further comprises a pressure channel adapted to monitor blood pressure within the ascending aorta.

35. The catheter system of claim 33, wherein the perfusion catheter comprises a plurality of perfusion catheters contained at least partially within the vehicle catheter.

36. The catheter system of claim 26, wherein each of the collecting catheter and the perfusion catheter further comprise an inflatable balloon disposed proximate a distal end.

37. The catheter system of claim 36, wherein the inflatable balloon is adapted to at least one of (i) substantially completely block a coronary ostium to isolate the coronary system from systemic circulation or (ii) are adapted to not completely block the coronary ostium.

38. The catheter system of claim 37, wherein, upon inflation, the inflatable balloon adapted to not completely block the coronary ostium comprises at least one of (i) a ring shape or (ii) a butterfly shape.

39. The catheter system of claim 26, wherein the perfusing collecting catheter further comprises:
a. a changeable curvature; and
b. a changeable tip angulation.

40. The catheter system of claim 39, wherein curvature and tip angulation may be changed implementing fibers along the length of the catheter to facilitate coronary engagement.

41. The catheter system of claim 26, wherein:
a. the collecting catheter is adapted to be engaged in the coronary sinus ostium to collect venous blood from the patient; and
b. the perfusion catheter is adapted to be engaged into the coronary artery ostia of the patient to deliver blood to the patient.

42. The catheter system of claim 26, wherein the perfusion catheter further comprises a sensor.

43. The catheter system of claim 42, wherein the sensor comprises:
a. a temperature sensor disposed proximate a wall of the tubular component; and
b. a pressure sensor disposed proximate the wall of the tubular component.

44. The catheter system of claim 26, wherein the perfusion catheter further comprises an inflation channel disposed proximate a wall of the tubular component.
First General Method: Coronary Closed Loop Dialysis

45. A method of coronary analysis, comprising:
a. obtaining access to a coronary access site in a patient via a predetermined entry site of the patient;
b. introducing an arterial sheath via the entry site;
c. introducing a collecting catheter into a first coronary artery via the arterial sheath to the entry site;
d. introducing a perfusion catheter into a second coronary artery via the arterial sheath to the entry site;
e. engaging a distal end of the perfusion catheter at an engaging site, the distal end of the perfrusion catheter comprising a pre-shaped curvature to facilitate engagement of the perfusion catheter at the entry site;
f. connecting the collecting catheter to an input of a multi-chambered dialysis machine;
g. connecting the perfusion catheter to an output of the multi-chambered dialysis machine;
h. collecting venous blood using the collecting catheter;
i. introducing the collected blood into a first chamber of the multi-chambered multi-chambered dialysis machine;
j. performing a first processing on the blood in the first chamber;
k. introducing processed blood from the first chamber into a second chamber of the multi-chambered multi-chambered dialysis machine;
l. performing a second processing on the blood in the second chamber; and
m. returning processed blood to the patient via the perfusion catheter.

46. The method of claim 45, wherein the arterial sheath is introduced using Seldinger's method.

47. The method of claim 45, wherein the entry site is at least one of (i) a femoral artery, (ii) a left subclavian vein to engage into the coronary sinus, (iii) a right subclavian vein into right atrium, (iv) a right jugular vein directly into right atrium, or (v) a femoral vein.

48. The method of claim 45, further comprising configuring the first chamber as a blood separation chamber for separating blood plasma and a harmful substance from the blood, the blood separation chamber adapted to provide at least one of (i) a plasmapheresis function or (ii) an aphaeresis function.

49. The method of claim 48, wherein the harmful substance comprises at least one of (i) LDL, (ii) fibrinogen, (iii) C reactive protein, (iv) cytokines, (v) cholesterol, (vi) triglyceride, (vii) perfused HDL, (viii) Lp (a), (ix) tissue factor, (x) interleukine, (xi) interleukine 1, (xii) interleukine 6, (xiii) TNF alpha, (xiv) a chemoattractant molecule, (xv) CD 14, (xvi) C3 complement, (xvii) C4 complement, or (xviii) C 1 inhibitor

50. The method of claim 45, wherein the second chamber is adapted to at least one of (i) deliver a drug into the blood, (ii) deliver a therapeutic component into the blood, (iii) maintain blood oxygenation at a desired level, (iv) sustain a desired coronary perfusion pressure, (v) or increase blood temperature to a desired level.

51. The method of claim 50, wherein the drug is a high level of drug.

52. The method of claim 50, wherein the therapeutic component is delivered continuously to the patient.

53. The method of claim 50, wherein the therapeutic component is at least one of (i) HDL, (ii) a statin, (iii) a gene, (iv) a chelating agent, (v) an anti-inflammatory agent, (vi) an angiotensin converting enzyme inhibitor, (vii) a peroxisome proliferator activated receptor agonist, (viii) apolipoprotein apoA1, (ix) mutated apolipoprotein apoA1, or (x) a gene for gene therapy.

54. The method of claim 45, further comprising providing a flow rate of at least around 150 ml/min for processed blood.

55. The method of claim 54, wherein the flow rate is between around 150 ml/min and around 250 ml/min for processed blood.

56. The method of claim 45, wherein the engaging site is at least one of (i) a coronary artery ostium or (ii) a coronary sinus ostium.

57. The method of claim 45, wherein an outcome of the return of processed blood is at least one of (i) stabilizing a vulnerable plaque, (ii) decreasing the lipid content of the vulnerable plaque, (iii) reducing inflammatory activity throughout the coronary system, (iv) decreasing the number of macrophages in the vulnerable plaque, or (v) increasing the number of smooth muscle cells in the vulnerable plaque.

58. The method of claim 45, wherein the patient has been diagnosed with at least one of (i) atherosclerosis, (ii) chronic coronary artery disease, (iii) acute coronary syndrome, (iv) unstable angina, or (v) acute myocardial infarction.

59. The method of claim 45, further comprising:
a. providing a vehicle catheter, the vehicle catheter comprising a substantially tubular portion and a preformed curvature at its distal end, the curvature being compatible with an aortic arch; and
b. introducing the vehicle catheter via the arterial sheath to the ascending aorta above a coronary ostium.

60. The method of claim 59, further comprising:
a. providing the vehicle catheter with an inflatable aortic root ring;
b. providing a fluid pathway from an inflator to the inflatable aortic root ring; and
c. inflating the inflatable aortic root ring after a perfusion dialysis catheter contained within the vehicle catheter is engaged into the coronary ostium.

61. The method of claim 45, further comprising:
a. providing an annulus proximate a distal tip of the perfusing catheter;
b. connecting the distal tip of the perfusing catheter to a vacuum source; and
c. generating a negative pressure at the annulus upon engaging the distal tip of the perfusing catheter into the coronary ostium, the pressure being sufficient to facilitate attachment and fixation of the perfusion catheter in the coronary ostium.

62. The method of claim 61, wherein the negative pressure is generated when the distal tip of the perfusing catheter is approximately 2 to 3 cm into the coronary ostium.

63. The method of claim 45, further comprising using the coronary dialysis system to increase a level of useful factors in the plasma, the useful factors beneficial for promoting at least one of (i) plaque stabilization or (ii) plaque regression.

64. The method of claim 45, wherein:
a. the collecting catheter further comprises an inflatable balloon disposed proximate a distal end of the collecting catheter; and
b. a coronary ostium is occluded by inflating the inflatable balloon to substantially completely isolate the coronary arteries from systemic circulation in order to prevent dilution of processed blood by the systemic blood.

65. The method of claim 64, wherein the second chamber further comprises:
a. an oxygenator; and
b. a pump.

66. The method of claim 64, further comprising maintaining a coronary perfusion pressure of between around 100 mm Hg to around 150 mmHg where the coronary artery is completely isolated from blood flow.
Second General Method: Coronary Open Loop Dialysis

67. A method of coronary dialysis, comprising:
a. providing a coronary dialysis system comprising a catheter system and a multi-chamber dialysis machine, the coronary dialysis system forming a pathway for a patient's blood;
b. collecting the blood;
c. draining the blood through an inlet of the multi-chamber dialysis machine;
d. passing the blood through a plurality of processing chambers in the multi-chamber dialysis machine;
e. processing the blood within the plurality of processing chambers to at least one of (i) remove a harmful substance in the blood or (ii) add an enriching substance to the blood; and
f. pumping the processed blood back to the patient's coronary circulation through an outlet of the dialysis machine via a perfusion catheter component of the catheter system.

68. The method of claim 67, wherein processing further comprises at least one of (i) filtering a substance from the blood or (ii) precipitating a substance from the blood.

69. The method of claim 67, wherein:
a. the enriching substance comprises a predetermined level of a therapeutic agent; and
b. the therapeutic agent is maintained in an intimate vicinity of coronary endothelial cells by preventing dilution of processed blood by the systemic circulation.

70. The method of claim 67, further comprising:
a. providing an annulus in the perfusing catheter proximate the distal tip of the perfusing catheter;
b. operatively connecting the distal tip to a vacuum source; and
c. generating a negative pressure at the annulus upon engaging the distal tip into the coronary ostium, the negative pressure being sufficient to facilitate attachment and fixation of the perfusion catheter in the coronary ostium.

71. The method of claim 70, wherein the negative pressure is generated when the distal tip is approximately 2 to 3 cm into the coronary ostium.

72. The method of claim 67, wherein:
a. the coronary system of the patient is not completely isolated with respect to blood flow; and
b. the multi-chambered dialysis machine comprises a blood separation chamber, an enricher, a heater, and a pump.

73. The method of claim 72, wherein the blood separation chamber further comprises at least one of (i) a plasmaphaeresis chamber or (ii) an aphaeresis chamber.

74. The method of claim 73, wherein:
a. the plasmaphaeresis chamber is in fluid communication with the inlet;
b. the aphaeresis chamber is in fluid communication with the plasmapharaersis chamber following the plasmapharaersis chamber;
c. the heater is in fluid communication with the aphaeresis chamber following the aphaeresis chamber;
d. the enricher is in fluid communication with the heater following the heating chamber; and
e. the pump is in fluid communication with the enricher following the enricher and the outlet.

75. The method of claim 67, wherein:
a. the collecting catheter further comprises an inflatable balloon disposed proximate a distal end of the collecting catheter; and
b. a coronary ostium is not completely occluded by the inflatable balloon;
c. whereby the coronary arteries are not substantially isolated from systemic circulation.

76. The method of claim 72, further comprising:
a. separating blood cells from blood plasma in the blood separation chamber;
b. separating a predetermined substance from the blood plasma in the blood separation chamber;
c. enriching the blood in the enricher with a therapeutic agent;
d. heating the blood to an appropriate level with the heater; and
e. pumping processed blood with the pump at a desired flow rate and blood pressure.

77. The method of claim 76, wherein the predetermined substance separated from the plasma in the blood separation chamber comprises at least one of (i) LDL, (ii) extra HDL, (iii) CRP, (iv) fibrinogen, (v) a plasma cytokine, (vi) a chelating agent, or (vii) a transgenic material.

78. The method of claim 76, wherein the predetermined substance separated from the plasma in the blood separation chamber is separated by at least one of (i) precipitation, (ii) filtration, or (iii) adsorption.

79. The method of claim 76, wherein therapeutic agent comprises at east one of (i) HDL, (ii) a chelating agent, (iii) a transgene, or (iv) a drug.
Third General Method: Systemic Dialysis

80. A method of blood dialysis, comprising:
a. providing a dialysis system comprising a catheter system and a multi-chamber dialysis machine, the dialysis system forming a pathway for a patient's blood;
b. collecting the blood from a non-coronary drain site;
c. draining the blood through an inlet of the multi-chamber dialysis machine;
d. passing the blood through a plurality of processing chambers in the multi-chamber dialysis machine;
e. processing the blood within the plurality of processing chambers to at least one of (i) remove a harmful substance in the blood or (ii) add an enriching substance to the blood; and
f. pumping the processed blood back to the patient's coronary circulation through an outlet of the dialysis machine via a perfusion catheter component of the catheter system into a non-coronary return site.

81. The method of claim 80, wherein the non-coronary drain site comprises a femoral artery and the non-coronary return site comprises a femoral vein.

82. The method of claim 80, wherein processing further comprises at least one of (i) filtering a substance from the blood or (ii) precipitating a substance from the blood.

83. The method of claim 80, wherein the enriching substance comprises a predetermined level of a therapeutic agent.

84. The method of claim 80, wherein the dialysis system multi-chambered dialysis machine comprises a blood separation chamber, an enricher, a heater, and a pump.

85. The method of claim 84, further comprising:
a. separating blood cells from blood plasma in the blood separation chamber;
b. separating a predetermined substance from the blood plasma in the blood separation chamber;
c. enriching the blood in the enricher with a therapeutic agent;
d. heating the blood to an desired level with the heater; and
e. pumping processed blood in the pump at a desired flow rate and blood pressure.

86. The method of claim 85, wherein the predetermined substance separated from the plasma in the blood separation chamber comprises at least one of (i) LDL, (ii) extra HDL, (iii) CRP, (iv) fibrinogen, (v) a plasma cytokine, (vi) a chelating agent, or (vii) a transgenic material.

87. The method of claim 85, wherein the predetermined substance separated from the plasma in the blood separation chamber is separated by at least one of (i) precipitation, (ii) filtration, or (iii) adsorption.

88. The method of claim 85, wherein therapeutic agent comprises at east one of (i) HDL, (ii) a chelating agent, (iii) a transgene, or (iv) a drug.

89. The method of claim 84, wherein the blood separation chamber further comprises at least one of (i) a plasmaphaeresis chamber or (ii) an aphaeresis chamber.

90. The method of claim 89, wherein:
a. the plasmaphaeresis chamber is in fluid communication with the inlet;
b. the aphaeresis chamber is in fluid communication with the plasmapharaersis chamber following the plasmapharaersis chamber;
c. the heater is in fluid communication with the blood separation chamber following the blood separation chamber;
d. the enricher is in fluid communication with the heater following the heater; and
e. the pump is in fluid communication with the enricher following the enricher and the outlet.
Fourth General Method: Pericardial Dialysis

91. A method of fluid dialysis, comprising:
a. providing a dialysis system comprising a catheter system and a multi-chamber dialysis machine, the dialysis system forming a pathway for a patient's blood;
b. introducing a collection catheter into a pericardium of a patient;
c. collecting fluid from within the pericardium;
d. draining the fluid through an inlet of the multi-chamber dialysis machine;
e. passing the fluid through a plurality of processing chambers in the multi-chamber dialysis machine;
f. processing the fluid within the plurality of processing chambers to at least one of (i) remove at least one harmful substance in the fluid or (ii) add at least one enriching substance to the fluid; and
g. pumping the processed fluid back to the pericardium through an outlet of the dialysis machine via a perfusion catheter component of the catheter system into the pericardium.

92. The method of claim 91, wherein processing further comprises at least one of (i) filtering a substance from the fluid or (ii) precipitating a substance from the fluid.

93. The method of claim 91, wherein the enriching substance comprises a predetermined level of a therapeutic agent.

94. The method of claim 91, wherein the dialysis system multi-chambered dialysis machine comprises a fluid component separation chamber, an enricher, a heater, and a pump.

95. The method of claim 94, further comprising:
a. separating a predetermined substance from the fluid in the fluid component separation chamber;
b. enriching the fluid in the enricher with a therapeutic agent;
c. heating the fluid to an desired level with the heater; and
d. pumping processed fluid in the pump at a desired flow rate and pressure.
